(19) United States
(12) Patent Application Publication
(10) Pub. No.: US 2024/0282422 A1
(43) Pub. Date: Aug. 22, 2024

(54) METHOD AND SYSTEM FOR ACTIVATING AND REACTIVATING A PRESCRIPTION

(71) Applicant: BLINK HEALTH INC., New York, NY (US)

(72) Inventors: Michael VANCE, Lake Forest Park, WA (US); Geoffrey CHAIKEN, Bellevue, WA (US); Matthew CHAIKEN, New York, NY (US)

(73) Assignee: BLINK HEALTH INC., New York, NY (US)

(21) Appl. No.: 18/173,003

(22) Filed: Feb. 22, 2023

(51) Int. Cl. G16H 20/10; G16H 80/00

(52) U.S. Cl. CPC G16H 20/10; G16H 80/00

(57) ABSTRACT

This application includes a method for activating or reactivating a prescription, which includes: receiving, over a network, customer information and prescription information of one or more customers; receiving, over the network, claim responses based on the customer information and prescription information, to submitted prescription claims for each of the one or more customers from at least one coverage provider; determining adjusted costs of the prescriptions based on initial prescription costs and on determined available discounts applicable to the prescriptions; performing a suitability check based on the claim responses and the adjusted costs to generate a set of suitable customers; and transmitting customer communications to the suitable customers for activation or reactivation, wherein the customer communications include adjusted costs prescriptions.

### TECHNICAL FIELD

The present invention relates generally to a network system and method for activating and reactivating prescriptions and more particularly to activating a new prescription or reactivating lapsed prescriptions in connection with a third-party system over a network connection.

### BACKGROUND

Between 20-50% of all new prescriptions that are written never get filled by patients. The failure to fill prescriptions and the failure to then adhere to taking prescribed medication costs an estimated $105 billion per year in the United States. Further, the failure to fill and adhere to prescribed medications leads to shorter life spans and lower productivity in the workforce.

Traditional retail pharmacies lack the infrastructure to support patient's either starting or continuing their medications. Further, the current way patients are treated leads to patients' data being decentralized into numerous brick and mortar retail pharmacies exacerbates this lack of infrastructure to engage with patients. Additionally, traditional retail pharmacies lack the additional infrastructure or capability to maintain and ensure compliance with laws, regulations, and/or other third-party requirements for each prescription written or filled.

Many customers that receive prescriptions fail to start, renew, or refill their prescriptions for a variety of reasons. The reasons for such lapses are varied and include failing to refill due to cost, inconvenience, or forgetfulness.

When a customer purchases a prescription product, such as a medication or medical device, an insurance or medical or prescription benefit plan may cover part of the cost. As a commercially insured customer, the customer will often have to contribute some amount of the purchase price in the form of a copay or deductible. Additionally, customers without insurance coverage may have to pay the full retail price for the prescription out of pocket. This presents a financial hurdle to many customers. In addition, customers are typically inconvenienced when purchasing a prescription because such purchase requires travel to and from a pharmacy, waiting on lines that are typically present at many pharmacies, and unknown medication pricing and insurance coverage before reaching the physical location of the pharmacy.

Many manufacturers of prescription products offer discount or assistance programs to help a customer, including the commercially insured customer, pay for their prescriptions. These programs can include many types of assistance including coupons, vouchers, refunds, and rebates that cover part or all the out-of-pocket cost of the product. Certain prescriptions are eligible for discounts when paid for in cash rather than through insurance coverage. In some cases, the discount can cover all or part of the out-of-pocket cost of the prescription for a customer with or without primary prescription coverage. However, many customers are unaware of these various saving opportunities. Determining the lowest price for a medication for a particular customer can involve gathering data from their insurance and/or pharmacy benefit manager companies, the relevant drug manufacturer, and foundations, which can be difficult, confusing, and time consuming.

Accordingly, a method for activating or reactivating a prescription, including for example a medical prescription and/or a drug benefit, while minimizing cost and increasing efficiency is desirable.

### BRIEF SUMMARY OF THE INVENTION

In accordance with one embodiment of the disclosure, the disclosure includes a method for activating or reactivating a prescription, comprising:
receiving, over a network, customer information and prescription information of one or more customers; submitting, over the network, based on the customer information and prescription information, a prescription claim for a prescription for each of the one or more customers to at least one coverage provider; receiving claim responses to the submitted prescription claims; determining adjusted costs of the prescriptions based on initial prescription costs and on available discounts applicable to the prescriptions; performing a suitability check based on the claim responses and the adjusted costs to generate a set of suitable customers; and transmitting customer communications to the suitable customers for activation or reactivation, wherein the customer communications include adjusted costs prescriptions.

In accordance with one or more embodiments, systems, and methods for activating or reactivating a prescription is provided. In one embodiment, the method includes receiving, over a network, data feed(s) from one, two, or a plurality or a network of pharmacies, standardizing the data feed(s) from each pharmacy into a standardized data feed of customer and prescription information for one customer or a plurality of customers. A suitability check is then performed on each, or a subset, of the plurality of customers to determine if a customer is, for example, (a) covered by insurance, (b) what amount (if any) of the prescription may be covered by insurance and what amount is remaining to be paid by the customer, and (c) if the customer is eligible for an available discount. A customer communication may then be transmitted to each customer or a subset of customers, wherein the customer communication includes an adjusted cost of the prescription.

In one embodiment, performing a suitability check on each, or a subset, of the plurality of customers to generate a set of suitable customers includes determining a likelihood of activation or reactivation based on the claim response, the adjusted cost, the original and remaining number of refills, whether a customer will respond to other non-financial stimuli such as a call from a health care provider or educational material, and on other such customer information.

In a further embodiment, the method further includes receiving a customer response to the customer communication from a specific suitable customer, wherein the customer response includes affirming interest in activating or reactivating prescription and a purchase payment for the prescription, creating a customer record for the specific suitable customer, and transmitting a fulfillment communication to the third party over the network to cause the prescription of the suitable customer to be processed by a dispensing system of the third party.

These and other advantages of the invention will be apparent to those of ordinary skill in the art by reference to the following detailed description and the accompanying drawings.

### BRIEF DESCRIPTION OF THE DRAWINGS

FIG. 1 is a block diagram of a network environment for activating or reactivating a prescription for suitable customers according to an embodiment.

FIG. 2 is a flowchart of a method for determining suitable customers and transmitting a customer communication regarding reactivation according to an embodiment.

FIG. 3 is a flow diagram illustrating training and operation of a machine learning model, according to an embodiment of the disclosure.

FIG. 4 is a flowchart of a method for activation or reactivation and payment for a prescription according to an embodiment.

FIG. 5 is a high-level block diagram of a computer system for implementing the systems and methods described herein.

### DETAILED DESCRIPTION

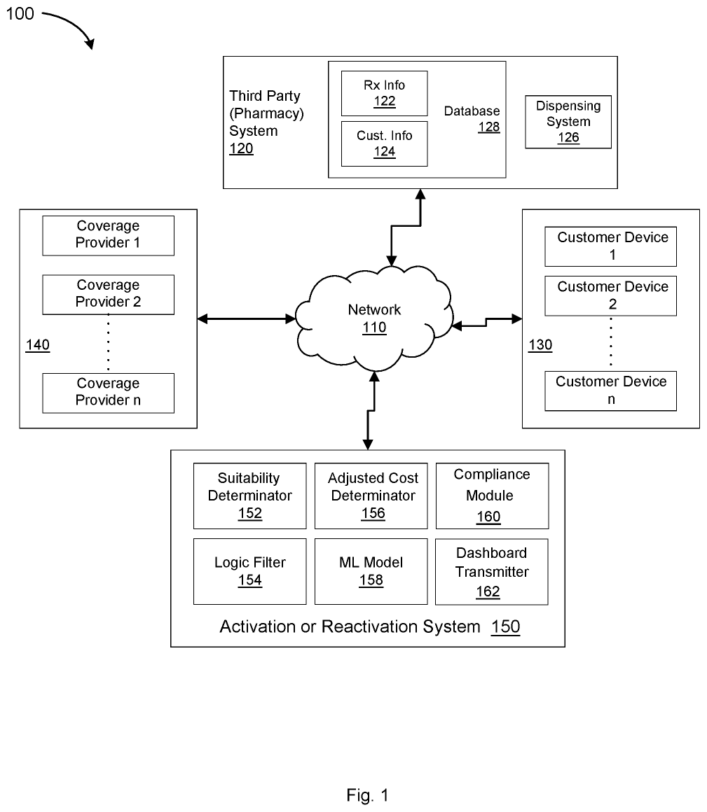

FIG. 1 is a block diagram of a network environment 100 for activating or reactivating a prescription for suitable customer(s) according to an embodiment. In use of network environment 100, the number of customer(s) can vary substantially, from 1 customer, to a substantially unlimited upper bound based on operating hardware. During use of network environment 100, the number of customer(s) the network environment 100 is configured to interact with will be about 500 customers or more, about 1,000 customers or more, about 10,000 customers or more, about 50,000 customers or more, about 100,000 customers or more, about 500,000 customers or more, about 1,000,000 customers or more, about 5,000,000 customers or more, about 10,000,000 customers or more, about 50,000,000 customers or more, about 100,000,000 customers or more, with even higher numbers being within the capability of network environment 100.

The network environment 100 includes a network 110 connected to one or more third-party systems 120, one or more customer devices 130, one or more coverage providers 140, and an activation or reactivation system 150.

Referring to FIG. 1 in more detail, the network 110 is a network for communication between participant devices and systems. An illustrative example network 110 is the Internet. The network 110 may be composed of multiple connected sub-networks or autonomous networks. The network 110 can be a local-area network (“LAN”), such as a corporate intranet, a metropolitan area network (“MAN”), a wide area network (“WAN”), an inter-network such as the Internet, a virtualized network, or a peer-to-peer network; e.g., an ad hoc WiFi peer-to-peer network. The network 110 may be composed of multiple connected sub-networks or autonomous networks. In some implementations, a wireless portion of the network 110 follows one of the IEEE 802.11 standards or wireless communications standards, such as WiMax, HSPA, LTE, etc. Any type and/or form of data network and/or communication network can be used for the network 110. It can be public, private, or a combination of public and private networks. In general, the network 110 is used to convey information between devices; e.g., for communication between the third-party system(s) 120, the one or more customer devices 130, the one or more coverage providers 140, and the activation or reactivation system 150.

The third-party system 120 is a system of a third party or multiple third parties, such as a pharmacy, that includes a database 128 storing prescription information 122 (and also optionally the prescription or script itself) and customer information 124, and a dispensing system 126 configured to dispense prescriptions. This third-party system 120 can be a system of a single pharmacy or a single location capable of providing medication(s), or this third-party system 120 can be a network of one or more pharmacies or one or more locations capable of providing medication(s).

The prescription information 122 includes data and metadata related to a prescription, or a script, e.g., for a medical device or a medication. In an embodiment, the prescription information 122 includes a type and name of a medical device or medication, the dosage amount, a quantity of medication per fill, and the number of remaining refills of the prescription, for example.

The prescription information 122 is received from a health care provider (not shown), e.g., a primary care physician or an emergency medical care professional. In one embodiment, the prescription information is provided via a phone call between the health care provider and the third-party pharmacy. In a further embodiment, the third-party system 120 is configured to receive and manage electronic prescriptions from health care providers such as doctors, dentists, physician assistants, nurse practitioners, hubs, and other medical professionals licensed to issue prescriptions through network 110 or any other suitable network. In some implementations, the third-party pharmacy receives prescriptions from third-party medical facilities (e.g., hospitals, clinics, and other medical service providers) via fax. The prescription information 122, as well as the customer information 124 is stored within the database 128 of the third-party system 120.

The customer information 124 stored within the database 128 of the third-party system 120 includes data related to a customer, including any or all of the following data such as customer name, address, date of birth, associated medical professionals, insurance information, communication information and/or preferences, and/or additional information.

The dispensing system 126 is a system within the third-party system 120 that is configured to access the prescription information 122 and customer information 124 from the database 128 so that a prescription order of a customer can be prepared. The dispensing system 126 can also receive transmissions directly through the network 110 from, such as, the activation or reactivation system 150. In an embodiment, the dispensing system 126 is one or more humans and/or one or more devices and/or one or more machines (including those with robotic components), capable of preparing, wholly or partially, a prescription product for receipt by a customer, by placing pills in bottles, mixing creams, etc. so that the prescription product is ready to be provided to a customer.

The customer device, e.g., customer device 1, customer device 2, and customer device n (hereinafter referenced in the singular as customer device 130 or in the plural as customer devices 130) is a computing or communication tool used by a customer. In this embodiment, the customer can include the patient the prescription information 122 pertains to, that patient's guardian or family member, and/or a caretaker or aide acting on behalf of that patient. The customer device 130 can be any suitable device that is capable of wired or wireless data transmission, such as but not limited to a personal computing device such as a smartphone, a tablet, a notebook, laptop computer, or a desktop computer. In some implementations, the customer device 130 includes one or more of: a processor, a display, a speaker, a microphone, and/or a camera. In some instances, a customer may use multiple customer devices 130 for different interactions described herein. In an embodiment described below, the customer device 130 receives a communication such as an email and transmits a response such as a completed website order and associated payment information.

The coverage provider, e.g., coverage provider 1, coverage provider 2, and coverage provider n (hereinafter referenced in the singular as coverage provider 140 or in the plural as coverage providers 140) is a provider of coverage for any item that could be subject of the prescription information 122. In an embodiment, the coverage provider 140 is an insurance company. The third-party system 120 may include customer information 124 related to customers that are provided coverage from coverage provider 1, customers that are provided coverage from coverage provider 2, and customers that are provided coverage from coverage provider n. Additionally, a single customer may be covered or associated with multiple insurance companies, e.g., coverage provider 1 through coverage provider n.

The activation or reactivation system 150 is a system configured to access the third-party system 120 via the network 110 and receive prescription information 122 and customer information 124 of one customer or a plurality of customers in order to determine a set of suitable customers. In an embodiment, the activation or reactivation system 150 is further configured to receive a customer response including affirming interest in activating or reactivating a prescription and optionally a purchase payment.

The activation or reactivation system 150 can then transmit a fulfillment communication, which can include a transmission of the purchase payment if one is received by the activation or reactivation system 150, to the third party system 120 so that the dispensing system 126 can dispense the prescription.

In one embodiment, the activation or reactivation system 150 includes a suitability determinator 152, a logic filter 154, an adjusted cost determinator 156, a machine learning model 158, an optional compliance module 160, and an optional dashboard transmitter 162. The activation or reactivation system 150 performs methods further described below.

The suitability determinator 152 is configured to perform a suitability check on each of a plurality of customers from a list of customers received from the third-party system 120 via the network 110. The suitability check is based on, e.g., which prescriptions are covered by a coverage provider 140, coverage dates of the prescription, whether generic versions of prescription medications or items are available, whether suitable replacement prescription medications or items are available, guidelines of medications or other items listed in their prescription, demographic data of the customer, market availability of the medication or other item listed in their prescription (including, e.g., dosage, formulation, and quantity and whether the particular medication is recalled or is to be replaced for use by an alternative medication), medication or other item interactions including medications prescribed since the patient's previous refill, address changes since the patients last refill, and on a determined adjusted cost for a prescription.

In some embodiments, coverage of a prescription by a coverage provider 140 may be determined by the activation or reactivation system 150 by submitting a prescription claim to one or more coverage providers 140 and receiving a claim response. Whether a prescription is covered, and how much of the cost is covered, is determined based on the coverage provider 140, the plan a customer is enrolled in, the type of medication prescribed in the prescription, and the like. In other embodiments, the activation or reactivation system 150 does not determine coverage of a prescription by a coverage provider, such as when coverage is already known, or can be estimated by, the activation or reactivation system 150.

The activation or reactivation system 150 can also receive notifications of additional steps that may, should, or must be performed prior to transmitting, through network 110, customer communications to the suitable customers for activation or reactivation. These additional steps can include, for example, the need for prior authorization from an insurance company, and/or clinical data needed. The activation or reactivation system 150 can then automatically route, if present, the one or more additional steps to either additional automated processes or to humans who conduct a task (such as calling or contacting an entity to procure a prior authorization (PA)). Once a task is completed, whether by automation or by human, the process then proceeds to the next step. Once all steps are performed the system transmits a command to a third-party pharmacy system to dispense and fulfill the prescription.

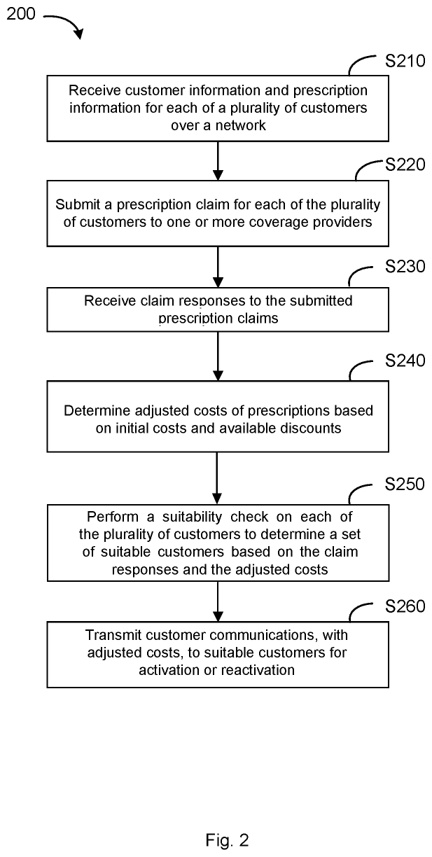

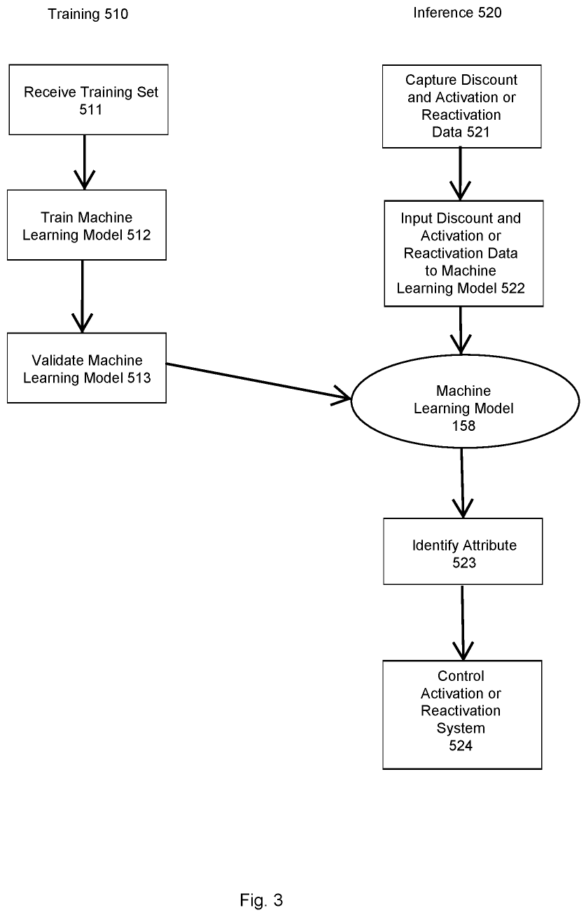

A prescription has an initial cost, which is the undiscounted retail price of the prescription, and an adjusted cost. The difference between the initial cost and the adjusted cost of a prescription is a savings amount that can be used to help determine if a customer will likely respond positively to a customer communication. The method for performing the claim submission, determining an adjusted cost, performing a suitability check, transmitting a customer communication to reactivate a prescription, and receiving a response from the customer is further explained in the flowcharts shown in FIGS. 2 and 3. FIGS. 2 and 3 will be described in conjunction with FIG. 1.

The optional compliance module 160 can operate in conjunction with the MLM 158 so that certain government and/or coverage provider 140 specific rules and/or certain third party pharmacy system 120 rules are taken into account. The optional compliance module 160 could influence MLM 158 operations as well as any other outputs of the activation or reactivation system 150. The optional compliance module 160 can access a database within the activation or reactivation system 150, and/or the optional compliance module 160 can actively contact third party rules databases, and/or the optional compliance module 160 can update a database within the activation or reactivation system 150 based on communications and transmissions received by other customers.

For example, the optional compliance module 160 can access and take into account federal laws regarding customers who are within certain government programs, such as Medicare and Medicaid. As another example, the optional compliance module 160 can access and take into account certain rules created by certain coverage providers 140. Certain communications or contacts from the activation or reactivation system 150 could then be allowed, modified, or prevented from transmission from the activation or reactivation system 150.

The optional dashboard transmitter 162 can be a module or portion of any component of the activation or reactivation system 150, or a separate hardware element that is capable of wired or wireless data transmission with the activation or reactivation system 150.

The optional dashboard transmitter 162 can provide an outputted data feed, through network 110, to parties that have access to various data received and/or transmitted by the activation or reactivation system 150. For example, one or more third parties, such as a pharmaceutical manufacturer, and/or one or more third party pharmacy systems 120 can receive output from the optional dashboard transmitter 162. Output of the optional dashboard transmitter 162 can then be viewed by the party in any suitable format, including text and/or image(s) to convey selected data.

The optional dashboard transmitter 162 can also receive input, through network 110, by parties that can transmit input to the activation or reactivation system 150. For example, one or more third parties, such as a pharmaceutical manufacturer, and/or one or more third party pharmacy systems 120 can transmit data that is received by the optional dashboard transmitter 162.

As one example of the optional dashboard transmitter 162, an output from the ML model 158 of a certain expected probability of activation or reactivation for one or more customers can be viewed, with the associated, customized, available discount expected to reach that probability. In this example, a third party, such as a pharmaceutical manufacturer, may modify one or both of the probability and/or the expected cost to achieve the customized, available discount, and receive updated data about how that modification alters estimated outcomes. The third party, such as a pharmaceutical manufacturer, can view such data by any suitable stratification, such as all (or a subset of) customers with a prescription for a certain medication, and/or all (or a subset of) customers in a certain geographic area, and/or all (or a subset of) customers by their associated demographic information.

Data received through the dashboard transmitter 162 can be used by the activation or reactivation system 150 to impact any other element of the activation or reactivation system 150, and impact resultant data sent by the activation or reactivation system 150, through network 110, to customer devices 130 and/or third party pharmacy system 120 and/or coverage providers 140. For example, upon receipt of a selection of a subset of customers for a certain medication, and an estimated customized, available discount of $8 resulting in an expected probability of activation or reactivation within 4 days of 60%, the activation or reactivation system 150 can automatically (and/or upon receipt of an allowing instruction by an operator of the activation or reactivation system 150) transmit customer communications to customer devices 130.

FIG. 2 is a flowchart of a method for activating or reactivating a prescription for suitable customers according to an embodiment. The method 200 begins at step S210 where customer information and prescription information for a plurality of customers is received over a network. In an embodiment, the customer information and prescription information are received from third party system 120 via network 110. The customer information includes data and metadata relating to each of a plurality of customers and prescriptions that have been prescribed to each of the plurality of customers. The data may include names, addresses, dates of birth, associated medical professionals and additional information. The prescription information is data and metadata related to a prescription for each of the plurality of customers and may include a type and name of a medication, a dosage amount, a quantity of medication per fill, and the number of remaining refills of the prescription.

In some embodiments, the method 200 proceeds directly from S210 to S240. In other embodiments, the method 200 proceeds directly from S210 to S220, thus, S220 is optional and does not need to occur for each prescription claim for each of the plurality of customers, such as when claim responses and/or discounts are known or can be estimated. At optional step S220, a prescription claim for each of the plurality of customers is submitted, over the network, to at least one coverage provider 140 for a prescription. A coverage provider can be an insurance company or other provider of prescription coverage.

In some embodiments, the method 200 proceeds directly from S210 to S240. In other embodiments, the method 200 proceeds directly from S210 to S220, and then from S220 to S230, thus, S220 and S230 are optional and do not need to occur for each prescription claim for each of the plurality of customers, such as when claim responses and/or discounts are known or can be estimated. At optional steps S220 and S230, claim responses to the prescription claims submitted in step S220 are received from the coverage provider 140.

At step S240, adjusted costs of the prescriptions for each of the plurality of customers are determined by an adjusted cost determinator 156, which can be configured to operate in conjunction with the MLM 158. The adjusted cost is based on an initial cost of the prescription and on determined available discounts applicable to the prescription and can be referred to herein as the customized available discount, and can be specific for each individual customer, or can be specific for a group of customers. This determination of the adjusted costs of the prescriptions for each of the plurality of customers can occur one or more times per customer over time, or this determination of the adjusted costs of the prescriptions for each of the plurality of customers can occur for each prescription each customer has and/or each time that same customer activates or reactivates that prescription over time. The discounts include discounts provided for prescriptions that are paid in cash rather than through insurance coverage, manufacturers or retailers coupons, refunds, rebates that cover part or all the out-of-pocket cost of the medical product, and bulk discounts that are negotiated between a medication manufacturer and a third party. The available discounts can be a standard discount that applies to all potential, suitable customers, or the available discounts can be customized based on those particular customer's suitability discussed above (such as that customer's previous activation or reactivation history, or similar customer(s) activation or reactivation history), which can be based on an output of the MLM 158, such as a suggested refund and/or rebate and a predicted probability of activation or reactivation based on past behavior of that customer and/or a plurality of other customers.

As one example of a customized available discount, the MLM 158 can compare one customer or a plurality of customers with another set of customers that have certain similarities, such as geographic location, age, other known prescriptions, etc. and can determine that a refund and/or rebate of $10 will likely increase the activation or reactivation of that customer's prescription information by 40% as compared to refunds and/or rebates of zero dollars. This comparison can occur based on the data feed received through network 110, which can then be standardized to a standardized data feed and stored within the activation or reactivation system 150. Due to the various formats and types of data received by the activation or reactivation system 150 through the network 110, a standardization process can be used so that operations of the activation or reactivation system 150 can occur with substantially homogenous, standardized data. This standardization process can be any suitable process utilizing one or more suitable application programming interfaces (APIs) to modify fields and/or content of a received data feed to comply or substantially comply with the fields and/or content of the standardized data feed. Consequently, upon transmission out of the activation or reactivation system 150, that standardized data can be reverted back to the fields and/or content of the particular outgoing data feed through any suitable reversion method, such as using a suitable API.

This standardized data feed can be utilized by the MLM 158 and filtered, so that one or more customers of the same coverage provider 1 (or one or more customers of different coverage providers 140) are compared. This standardized data feed can be utilized by the MLM 158 and filtered, so that one or more customers of the same third party system 120 (e.g. customers of one branch or several branches of a pharmacy) can be compared, or that one or more customers of different third party systems 120 (e.g. customers of singular batches of several batches of pharmacy 1 and pharmacy 2). The MLM 158 can also be updated at real-time, or substantially real-time, with data as the standardized data feed is updated and/or added to. Thus, considerations and outputs of the MLM 158 can be continually, or substantially continually, be updated throughout many typical days of operation based on updated data feeds that are standardized into the standardized data feed and/or customer(s) real-time or substantially real-time activation(s) and reactivation(s).

At step S250, a suitability check is performed on each of the plurality of customers, or a subset thereof, based on the adjusted costs to determine which of the plurality of customers are suitable customers (and optionally the claim responses if steps S220 and S230 are performed). The step generates a set of suitable customers. In one embodiment, machine learning models may be used to determine the likelihood a customer would reactivate their prescription and then subsequently rank or list the plurality of customers in order of suitability scoring. For example, since the claims process incurs server load and cost, low-scoring prescriptions may not go through claims to generate a live price, but rather may generate alternative messaging to potential customers. Medium-scoring prescription information may receive an offer via email or other lower-cost communication method, while high-scoring prescriptions might get a phone call. These machine learning prefilters could be used in conjunction with prescription origin date, frequency of refills, total number of refills, last-filled date, demographic data, insurance status, cash and/or discount price, frequency of dosage, the chronic nature of the disease, and other relevant data. In another embodiment, suitable customers are sorted and given priority depending on the likelihood of the customer purchasing a prescription.

In addition to performing a suitability check, in one embodiment, step S250 may include determination of a likelihood of activation or reactivation for each of the suitable customers based on the claim response, the adjusted cost, and the customer information, which is optional, and can occur if steps S220 and S230 are performed. In an embodiment, determining a likelihood of activation or reactivation of a prescription is based on at least one of: a chronic or acute nature of a condition treated by the prescription, relevant drug interactions of the prescription with other prescriptions of the particular suitable customer (either input by that customer or their health care provider, and/or known to the activation or reactivation system 150 for the same customer), a remaining number of prescription refills, time elapsed since previous prescription refill, frequency at which previous refills were purchased, frequency of previous refills made by the same customer but for other prescriptions, activation or reactivation behavior of similar customer(s), a copay amount to be applied to the initial cost of the prescription, a deductible amount to be applied to the initial cost of the prescription, payment preferences of the particular suitable customer, changes in cost of the prescribed medication or item since last refill, and demographic factors of the particular suitable customer. In an embodiment, the likelihood of activation or reactivation by a customer is determined by a logic filter, e.g., logic filter 154 and/or the MLM 158 of FIG. 1, based on customer parameters.

For likelihood to activate or reactivate, the logic filter 154 and/or the MLM 158 would consider one or many sources of data, for example, for how long a customer adhered to the prescription and/or at what velocity. Adherence refers to the number of refills a customer has previously filled of their prescription. Velocity is the frequency of doses the customer has previously received. For example, if a customer's prescription is for four pills daily on a 120-pill prescription, then that customer can be estimated to fill an originally prescribed prescription, or a refill of that originally prescribed prescription, at a 30-day velocity. If that customer fills an originally prescribed prescription, or a refill of that originally prescribed prescription, at a 60-day velocity, then it could be estimated that the customer is at a velocity of 0.5. The logic filter 154 and/or the MLM 158 can base determinations on the combination of adherence, velocity, price, amongst other data, to learn what factors contribute to higher-likelihood prescriptions activations or reactivations. This learning is further described below.

At step S260, customer communications are transmitted to each of the suitable customers, or to a subset of suitable customers, for activation or reactivation of the prescription. The customer communications may include the adjusted costs of the prescriptions. The customer communications are transmitted using one or more communication options via the network 110, such as email, phone calls, SMS or text messages, targeted advertisement, push notifications, mailings, and the like. The customer communications may further include prescription delivery options, such as overnight delivery, standard shipping, or in-store pickup.

In an embodiment, a communication option is selected based on the received customer information, e.g., if it has previously been determined that an individual responds favorably to one type of communication compared to other types of communication. Additionally, the frequency of follow up communications and the contents of the communications can be tailored to a particular individual. In an embodiment, a machine learning model, e.g., the machine learning model (MLM) 158 of FIG. 1, is implemented to determine the type, frequency, and content of a customer communication that will result in the highest likelihood of engagement.

As used herein, the term “machine learning model” is meant to include a single machine learning model or an ensemble of machine learning models, which can optionally be used to at least partially perform one or more steps S240, S250 and S260. Each model in the ensemble may be trained to infer different attributes. The MLM 158 can be a program module of a processor of the activation or reactivation system 150 that performs the methods and functions described herein. The MLM 158 can be programmed into the integrated circuits of the processor.

The results output by the MLM 158 are data attributes, which are received as input by the one or more modules of the activation or reactivation system 150. The MLM 158 can be useful to predict data attributes that can be difficult or cumbersome to develop using more conventional approaches. For example, a customized available discount for a group of similar customers may be used an in put to the MLM 158, when then predicts various data attributes, such as expected activation or reactivation levels based on various discount levels. The activation or reactivation system 150 can then control the customized available discount transmitted to a specific customer device 1 of customer devices 130.

As described above, MLM 158 is especially useful to learn complex relationships and/or to automatically adapt to changes. FIG. 3 is a flow diagram illustrating training and operation of a machine learning model (MLM 158), according to an embodiment. The process includes two main phases: training 510 the MLM 158 and inference (operation) 520 of the MLM 158. These will be illustrated using an example where the machine learning model learns to predict the required increase in customized available discount to achieve a desired activation or reactivation level based on historical data of proposed discount levels and consequential activation or reactivation levels. The following example will use the term “machine learning model” but it should be understood that this is meant to also include an ensemble of machine learning models.

A training module (not shown) performs training 510 of the MLM 158. In some embodiments, the MLM 158 is defined by an architecture with a certain number of layers and nodes, with biases and weighted connections (parameters) between the nodes. During training 510, the training module determines the values of parameters (e.g., weights and biases) of the MLM 158, based on a set of training samples that include historical standardized data feeds of customer, prescription information and activation or reactivation information.

The training module receives a training set 511 for training the machine learning model in a supervised manner. Training sets typically are historical data sets of inputs of customized available discounts and corresponding responses of activation or reactivation. The training set samples the historical data sets of customized available discounts and corresponding responses of activation or reactivation operation, under varying or a wide range of different conditions. The corresponding responses are alterations of the customized available discounts and subsequent observations after some time interval, such as the actual activation or reactivation of one or more customers after an identified, customized available discount.

The following is an example of two training samples :
i. Customer A is Male, 54 years old, lives in Oregon, has fulfilled 1 out of a total of 4 refills, has not fulfilled in 6 weeks
ii. Received a customized available discount of $5 for a prescription of medication X
iii. has not reactivated the prescription within 7 days or receipt of customized available discount
i. Customer B is Female, 48 years old, lives in California, has fulfilled 1 out of a total of 4 refills, has not fulfilled in 6 weeks
ii. Received a customized available discount of $4 for a prescription of medication X
iii. has reactivated the prescription within 2 days or receipt of customized available discount

In these two examples, a customized available discount of $5 is not sufficient to effect reactivation of medication X by Customer A, but a customized available discount of $4 was sufficient to effect reactivation of medication X by Customer A. The MLM 158 can use these examples to learn that for other customers similarly situated to Customer B's facts as compared to Customer A, those other customers may have a higher likelihood of reactivation at a comparatively lower customized available discount.

In typical training 510, a training sample is presented as an input to the MLM 158, which then predicts an output for a particular attribute. The difference between the machine learning model's output and the known good output is used by the training module to adjust the values of the parameters (e.g., features, weights, or biases) in the MLM 158. This is repeated for many different training samples to improve the performance of the MLM 158 until the deviation between prediction and actual response is sufficiently reduced.

The training module also can validate 513 the trained MLM 158 based on additional validation samples. The validation samples are applied to quantify the accuracy of the MLM 158. The validation sample set includes additional samples of inputs of customized available discounts and corresponding responses of activation or reactivation. The output of the MLM 158 can be compared to the known ground truth. To evaluate the quality of the machine learning model, different types of metrics can be used depending on the type of the model and response.

Classification refers to predicting what something is, for example if an image in a video feed is a person. To evaluate classification models, F1 score may be used. The F1 score is a measure of predictive accuracy of a machine learning model. The F1 score is calculated from the precision and recall of the machine learning model, where the precision is the number of correctly identified positive results divided by the number of all positive results, including those not identified correctly, and the recall is the number of correctly identified positive results divided by the number of all samples that should have been identified as positive.

Regression often refers to predicting quantity, for example, how much energy is consumed. To evaluate regression models, coefficient of determination, which is a statistical measure of how well the regression predictions approximate the real data points, may be used. However, these are merely examples. Other metrics can also be used. In one embodiment, the training module trains the machine learning model until the occurrence of a stopping condition, such as the metric indicating that the model is sufficiently accurate or that a number of training rounds having taken place.

Training 510 of the MLM 158 can occur off-line, as part of the initial development and deployment of the activation or reactivation system 150. Under this option, training samples from historical customers can be used to train the MLM 158. This training data can be all available historical customer information, or a portion of the historical customer information for other customers that are similarly situated such as, for example, being the same/similar customized available discount, same/similar coverage provider 140, same/similar brand of third party system 120, same/similar age, same sex, same/similar geographic location, same/similar type of prescription, same/similar customer communication mode, same/similar velocity, same/similar communication history, same/similar stored preferences, same/similar historical engagement data such as open rate and click-through rates, etc.

The trained MLM 158 can then be deployed in the field. Once deployed, the MLM 158 can be continually trained 510 or updated. For example, the training module uses data captured in the field, during use of the activation or reactivation system 150, to further train the MLM 158. The training 510 can occur within the activation or reactivation system 150 and/or in an external database.

In operation 520, the MLM 158 uses the same inputs as input 522 to the MLM 158. The MLM 158 then predicts the corresponding response. In one approach, the MLM 158 calculates 523 a probability of possible different outcomes, for example the probability that a customer will reactivate within a prescribed amount of time. Based on the calculated probabilities, the MLM 158 identifies 523 which attribute is most likely. In a situation where there is not a clear cut winner, the MLM 158 may identify multiple attributes and seek verification, such as from a third party.

Continuing the above examples, Patient A is selected to receive an updated, customized available discount. The inputs to the MLM 158 are the following:
i. Customer A is Male, 54 years old, lives in Oregon, has fulfilled 1 out of a total of 4 refills, has not fulfilled in 7 weeks
ii. Received a customized available discount of $10 for a prescription of medication X

The MLM 158 predicts the following attributes 523: predicted increase of customized available discount from $5 to $10 increases probability that Customer A will reactivate within 7 days of receipt of customized available discount from 40% (when discount was $5) to 65% (when the discount is $10). The activation or reactivation system 150 can then transmit, through network 110, the updated, customized available discount to the customer device 130 by using the responses predicted by the MLM 158 to make informed decisions.

The activation or reactivation system 150 uses the MLM 158 to evaluate different possible courses of action. In this example, the MLM 158 functions as a simulation using an original, customized available discount, or one or more updated, customized available discounts, and can provide activation or reactivation probabilities for each of these simulations. The activation or reactivation system 150 can take different courses of action to affect a new customer, or a customer that has received one or more customized available discounts. For example, the activation or reactivation system 150 can increase the value of a first or subsequent customized available discounts, can determine what communication mode (telephone call, text, email, etc.), time of day to transmit the communication, etc.

A “policy” is a set of actions performed by the activation or reactivation system 150. In the above scenario, some example policies are as follows:
i. Policy 1: For customers that do not reactivate within 4 days of an original, customized available discount of a first offered value, an updated, customized available discount is to be sent at a second value, which is higher than the first value.
ii. Policy 2: For customers that do not reactivate within 4 days of an updated, customized available discount that was transmitted by email, a telephone call is to occur with that same, updated, customized available discount.

The policies can be a set of logic and rules determined by domain experts. They can also be learned by the activation or reactivation system 150 itself using reinforcement learning techniques. At each time step, the activation or reactivation system 150 evaluates the possible actions that it can take and can choose the action that maximizes evaluation metrics or provide an option for selection by a third party of an action. The activation or reactivation system 150 does so by simulating the possible subsequent states that may occur as a result of the current action taken, then evaluates how valuable it is to be in those subsequent states. For example, a valuable state can be that a customer reactivates within two days when presented with an original, customized available discount above a certain high value, rather than receive a lower value original, customized available discount followed by a higher value updated, customized available discount. This valuable state can be static or could change as the activation or reactivation system 150 learns that to achieve a certain probability of reactivation, that original, customized available discount above a certain high value should be transmitted before other customized available discounts.

Based on a goal or target cost and/or a goal or target probability of activation or reactivation, a policy engine of the activation or reactivation system 150 determines which polices might be applicable. This can be done using a rules-based approach, for example. The MLM 158 predicts the result of each policy. The different results are evaluated and a course of action is selected or provided for selection by others. A set of metrics is used to evaluate the policies.

Metrics can be defined to suit particular needs. For example, metrics to evaluate customers with various prescriptions. In one embodiment, the metrics can be defined to achieve an activation or reactivation probability of 85% for a specified customer or group of customers. Metrics can also be defined for different time horizons. For example, a policy may be chosen to optimize for immediate gains (higher original, customized available discount value to result in shorter term increase in probability of activation or reactivation), while another may be chosen to optimize for long-term benefits (lower, original customized available discounts but more frequent transmissions of step-wise increased value updated, customized available discounts).

To simulate subsequent states, the activation or reactivation system 150 uses the trained MLM 158. When underlying conditions (e.g. type of medication prescribed, number of medications prescribed, geographic location for the customer(s)) are changing, the MLM 158 can make predictions on what most likely will be observed as a result of actions taken. Based on these predictions, activation or reactivation system 150 chooses a policy or action that most likely maximizes the metric of interest, e.g. the probability of activation or reactivation being above a target threshold.

To decide which action to take from a state, the activation or reactivation system 150 may employ techniques of exploitation and exploration. Exploitation refers to utilizing known information. For example, a past sample shows that under certain conditions, a particular action was taken, and good results were achieved. The activation or reactivation system 150 may choose to exploit this information, and repeat this action if current conditions are similar to that of the past sample. Exploration refers to trying unexplored actions. With a pre-defined probability, the activation or reactivation system 150 may choose to try a new action. For example, 10% of the time, the activation or reactivation system may perform an action that it has not tried before but that may potentially achieve better results.

Returning to FIG. 1, a customer receives a customer communication on, and responds from, a customer device 130 where the response is sent back to the activation or reactivation system 150 via the network 110. The customer communication can be wholly or partially produced by any portion of the activation or reactivation system 150. For example the MLM 158 can produce ranked lists of customers to receive various customer communications via predicted or customer selected channels, with variations of the list being able to be created if no response is received back after a customer communication is sent. Additionally, any portion of the activation or reactivation system 150, for example the MLM 158, can review all customer communications for a particular customer and batch one or more customer communications together to avoid many multiple customer communications being received. This batching can occur based on total number of customer communications (e.g. number of medications), by specific coverage provider 140 and/or by specific third party pharmacy system 120.

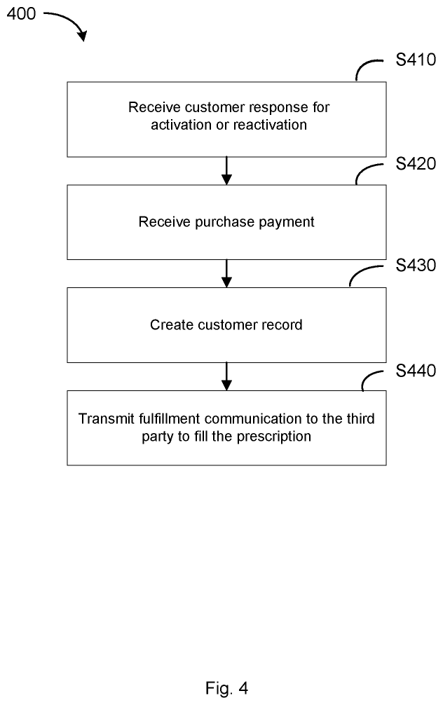

The process for activation or reactivation is further described in the method shown in FIG. 4. FIG. 4 is a flowchart of a method for activation or reactivation and payment for a prescription according to an embodiment. The method 400 is initiated when a suitable customer responds to the customer communication sent in step S260 of FIG. 2. At step S410 a customer response to the customer communications from the suitable customer is received. The customer response includes affirming interest in activating or reactivating the prescription and sending a purchase payment for the prescription. For example, if a suitable customer received a customer communication in the form of an email and clicked on a link to a website, the customer response may include selecting an option to reactivate a prescription and providing payment for any remaining refills via the linked website. In another example, if a suitable customer received a customer communication in the form of an email and clicked on a link to a website, the customer response may include selecting an option to engage with their health care provider in receiving a prescription that they can then purchase.

In some embodiments, the method 400 proceeds directly from S410 to S430, thus, S420 is optional and does not need to occur for each receipt of a customer response, such as when the customer submits their purchase payment directly to the third party pharmacy system 120 or directly to another party. At optional step S420, a purchase payment, if required, from the customer is received by the activation or reactivation system 150, alternatively or in addition to, the purchase payment from the customer is transmitted directly from the customer to the third party pharmacy system 120. The purchase payment may include a credit card charge, a payment from a bank account, a payment from a health savings account, and the like.

At step S430, a customer record for the particular suitable customer is created, as well as a record of where the purchase payment was transmitted. In this process a customer record of the purchase is created and stored in a database that is linked to the customer profile from the third-party pharmacy.

At step S440, a fulfillment communication is transmitted to the third party, e.g., via the network 110. For example, a message can be sent to a third-party pharmacy over a network to cause the prescription of the suitable customer to be processed by a dispensing system of the third-party pharmacy and prepare the prescription for pickup or delivery.

If the purchase payment was originally transmitted to the activation or reactivation system 150, subsequent to or concurrent to S440, that purchase payment can be transmitted from the activation or reactivation system 150 to the third party pharmacy system 120.

A last step of any method discussed herein can include either transmission of instructions to the customer and/or receipt of instructions by the customer for how to administer the prescription. For example, the transmission of instructions can occur through any suitable electronic means (e.g. text message, email, etc.) and/or through a phone call and/or in-person conversation between the customer and an employee of the third party pharmacy system 120. As an additional example, the instructions can include dosage for each administration and frequency of administration e.g. one pill by mouth 2 times a day, and/or 5 mL injection once a day. In other examples a last step of any method discussed herein can include administration of the prescription by the customer themselves, or a suitable third party. For example, the administration can include an administration of the prescription to the customer, e.g. a pill by mouth and/or an injection.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

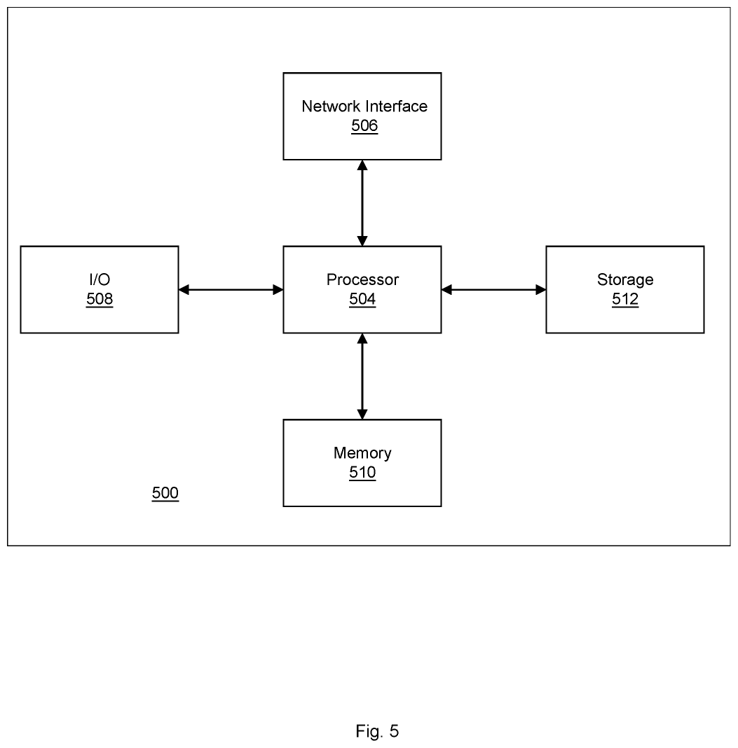

A high-level block diagram of an example computer 500 that may be used to implement systems, apparatus, and methods described herein is depicted in FIG. 5. Computer 500 includes a processor 504 operatively coupled to a data storage device 512 and a memory 510. Processor 504 controls the overall operation of computer 500 by executing computer program instructions that define such operations. The computer program instructions may be stored in data storage device 512, or other computer readable medium, and loaded into memory 510 when execution of the computer program instructions is desired. Thus, the method and workflow steps or functions of FIGS. 2-4 can be defined by the computer program instructions stored in memory 510 and/or data storage device 512 and controlled by processor 504 executing the computer program instructions. For example, the computer program instructions can be implemented as computer executable code programmed by one skilled in the art to perform the method and workflow steps or functions of FIGS. 2-4. Accordingly, by executing the computer program instructions, the processor 504 executes the method and workflow steps or functions of FIGS. 2-4. Computer 500 may also include one or more network interfaces 506 for communicating with other devices via a network. Computer 500 may also include one or more input/output devices 508 that enable user interaction with computer 500 (e.g., display, keyboard, mouse, speakers, buttons, etc.).

Processor 504 may include both general and special purpose microprocessors, and may be the sole processor or one of multiple processors of computer 500. Processor 504 may include one or more central processing units (CPUs), for example. Processor 504, data storage device 512, and/or memory 510 may include, be supplemented by, or incorporated in, one or more application-specific integrated circuits (ASICs) and/or one or more field programmable gate arrays (FPGAs).

Data storage device 512 and memory 510 each include a tangible non-transitory computer readable storage medium. Data storage device 512, and memory 510, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read- only memory (CD-ROM), digital versatile disc read-only memory (DVD-ROM) disks, or other non-volatile solid state storage devices.

Input/output devices 508 may include peripherals, such as a printer, scanner, display screen, etc. For example, input/output devices 508 may include a display device such as a cathode ray tube (CRT) or liquid crystal display (LCD) monitor for displaying information to the user, a keyboard, and a pointing device such as a mouse or a trackball by which the user can provide input to computer 500.

One skilled in the art will recognize that an implementation of an actual computer or computer system may have other structures and may contain other components as well, and that FIG. 5 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method for activating or reactivating a prescription, comprising:
receiving, over a network, customer information and prescription information of one or more customers;
determining adjusted costs of the prescriptions based on initial prescription costs and on determined available discounts applicable to the prescriptions;
performing a suitability check based on the adjusted costs to generate a set of suitable customers; and
transmitting customer communications to the suitable customers for activation or reactivation, wherein the customer communications include adjusted costs prescriptions.

2. The method of claim 1, further comprising, subsequent to receiving, over a network, customer information and prescription information of one or more customers,
submitting, over the network, based on the customer information and prescription information, the prescription claim for the prescription for each of the one or more customers to least one coverage provider; and
receiving, over the network, claim responses based on the customer information and prescription information, to submitted prescription claims for each of the one or more customers from the at least one coverage provider.

3. The method of claim 2, wherein performing a suitability check further comprises:
performing the suitability check based on the claim responses and the adjusted costs to generate the set of suitable customers.

4. The method of claim 2, wherein performing the suitability check further comprises:
determining a likelihood of activation or reactivation based on the claim responses, the adjusted cost, and the customer information.

5. The method of claim 4, wherein determining a likelihood of activation or reactivation is further based on at least one of: a chronic or acute nature of a condition treated by a prescription, relevant drug interactions of a prescription with other prescriptions of a suitable customer, a remaining number of prescription refills, a copay amount to be applied to the initial prescription costs, a deductible amount to be applied to the initial prescription costs, payment preferences of the suitable customers, and demographic factors of the suitable customers.

6. The method of claim 1, wherein the prescription information includes metadata related to a prescription, including at least one of: a type of medical device, a type of medication, a dosage amount, a quantity of medication per fill, and a number of remaining refills of the prescription.

7. The method of claim 1, wherein the customer communications are transmitted using one or more communication options, and wherein a communication option is selected based on the received customer information.

8. The method of claim 1, further comprising:
receiving customer responses to the customer communications from suitable customers, wherein the customer responses includes affirming interest in activating or reactivating prescriptions and a purchase payments for the prescriptions;
creating a customer record for each of the suitable customers; and
transmitting a fulfillment communication to a third party over the network to cause the prescriptions of the suitable customers to be processed by a dispensing system of the third party.

9. The method of claim 1, further comprising training a machine learning model using historical adjusted cost and suitability data.

10. The method of claim 1, further comprising generating a set of suitable customers and/or adjusted costs using a trained machine learning model.

11. A system for executing a method for activating or reactivating a prescription comprising:
a memory storing computer instructions; and
at least one processor configured to execute the computer instructions, the computer instructions configured to cause the at least one processor to perform operations of:
receiving, over a network, customer information and prescription information of one or more customers;
determining adjusted costs of the prescriptions based on initial prescription costs and on determined available discounts applicable to the prescriptions;
performing a suitability check based on the adjusted costs to generate a set of suitable customers; and
transmitting customer communications to the suitable customers for activation or reactivation, wherein the customer communications include adjusted costs prescriptions.

12. The system of claim 11, wherein the at least one processor subsequent to receiving, over a network, customer information and prescription information of one or more customers, is configured to perform the operation of:
submitting, over the network, based on the customer information and prescription information, the prescription claim for the prescription for each of the one or more customers to at least one coverage provider; and
receiving, over the network, claim responses based on the customer information and prescription information, to submitted prescription claims for each of the one or more customers from the at least one coverage provider.

13. The system of claim 12, wherein performing a suitability check further comprises:
performing a suitability check based on the claim responses and the adjusted costs to generate the set of suitable customers.

14. The system of claim 11, wherein performing a suitability check further comprises:
determining a likelihood of activation or reactivation based on the claim responses, the adjusted cost, and the customer information.

15. The system of claim 14, wherein determining a likelihood of activation or reactivation is further based on at least one of: a chronic or acute nature of a condition treated by a prescription, relevant drug interactions of a prescription with other prescriptions of a suitable customer, a remaining number of prescription refills, a copay amount to be applied to the initial prescription costs, a deductible amount to be applied to the initial prescription costs, payment preferences of the suitable customers, and demographic factors of the suitable customers.

16. The system of claim 11, the operations further comprising:
training a machine learning model using historical adjusted cost and suitability data. 17 The system of claim 11, the operations further comprising:
generating a set of suitable customers and/or adjusted costs using a trained machine learning model.

18. A non-transitory computer-readable medium storing computer program instructions for executing a method for activating or reactivating a prescription, the computer program instructions, when execution on at least one processor, cause the at least one processor to perform operations comprising:
receiving, over a network, customer information and prescription information of one or more customers;
determining adjusted costs of the prescriptions based on initial prescription costs and on determined available discounts applicable to the prescriptions;
performing a suitability check based on the claim responses and the adjusted costs to generate a set of suitable customers; and
transmitting customer communications to the suitable customers for activation or reactivation, wherein the customer communications include adjusted costs prescriptions.

19. The non-transitory computer-readable medium of claim 18, wherein the at least one processor subsequent to receiving, over a network, customer information and prescription information of one or more customers, is configured to perform the operation of:
submitting, over the network, based on the customer information and prescription information, the prescription claim for the prescription for each of the one or more customers to at least one coverage provider; and
receiving, over the network, claim responses based on the customer information and prescription information, to submitted prescription claims for each of the one or more customers from the at least one coverage provider.

20. The non-transitory computer-readable medium of claim 19, wherein performing a suitability check further comprises:
determining a likelihood of activation or reactivation based on the claim responses, the adjusted cost, and the customer information.

21. The non-transitory computer-readable medium of claim 18, the operations further comprising:
training a machine learning model using historical adjusted cost and suitability data.

22. The non-transitory computer-readable medium of claim 18, the operations further comprising:
generating a set of suitable customers and/or adjusted costs using a trained machine learning model.
